(19) United States
(12) Patent Application Publication
(10) Pub. No.: US 2026/0196356 A1
(43) Pub. Date: Jul. 9, 2026

(54) Systems and Methods for Governed Release of Clinical Outputs Generated by Automated Reasoning

(71) Applicant: OneSource Solutions International, Inc., Sudbury, MA (US)

(72) Inventors: Harold Arkoff, Sudbury, MA (US); Vedran Jukic, Trieste (IT)

(73) Assignee: OneSource Solutions International, Inc., Sudbury, MA (US)

(21) Appl. No.: 19/439,917

(22) Filed: Jan. 5, 2026

Related U.S. Application Data
(60) Provisional application No. 63/780,190, filed on Mar. 29, 2025.
(60) Provisional application No. 63/742,081, filed on Jan. 6, 2025.

(51) Int. Cl. G16H 50/30; G06F 21/16; G06F 21/32; G06F 21/60

(52) U.S. Cl. CPC G16H 50/30; G06F 21/16; G06F 21/32; G06F 21/602

(57) ABSTRACT

An AI keystone system for medical decision support generates governed, auditable clinical insights. A medical data governance (MDG) engine ingests heterogeneous patient-related data from clinical systems, medical devices, and clinician inputs, validates and normalizes the data under governance rules, and emits structured, context-constrained representations for identified patients and episodes. One or more intermediary components collect and relay real-time device data so downstream processing operates only on governed data. A reasoning layer with artificial-intelligence models, including at least one large language model, cooperates with modular AI components to process clinician queries and generate candidate clinical outputs. A cryptographic governance subsystem evaluates candidate outputs against patient-safety rules, regulatory mandates, institution-specific policies, and stored consent artefacts using machine-verifiable constraints, enforces dual-factor clinician authentication, and maintains immutable, hash-chained audit trails with trusted timestamps that gate workflow progression. The system enables defensible use of AI-assisted clinical insights across hospital, clinic, and virtual-care environments.

### CROSS-REFERENCE TO RELATED APPLICATIONS

This application claims the benefit of priority under 35 U.S.C. § 119(e) to U.S. Provisional Application No. 63/742,081, filed January 6, 2025, titled “AI Keystone System for Advanced Medical Decision Support and Scalable On-Demand Clinical Insights,” the entire contents of which are incorporated herein by reference and U.S. Provisional Application 63/780,190, filed March 29, 2025 titled “System and Method for Real-Time Clinical Insight Using AI-Driven Medical Data Governance.”

This application is further related to the following U.S. patents and applications, the entire disclosures of which are incorporated herein by reference:

U. S Non- Provisional Application No. 19/271,679, filed July 16, 2025, titled “Personalized AI Agent as a Case Manager,” the entire disclosure of which is incorporated herein by reference.

U.S. Provisional Application No. 62/804,838, filed February 13, 2019, titled “Cable/Dongle System for Connecting Medical Devices to a Display":

U.S. Patent No. 11,309,665 B1, issued April 19, 2022, titled “Active Cable Arrangement for Connecting Medical Devices to a Display”

U.S.Patent No. 11,693,990 B1, issued July 4, 2023, titled “Medical Data Governance,"and

U.S. Patent No. 12,001,464 B1, issued June 4, 2024, titled “System and Method for Medical Data Governance Using Large Language Models.”

### FIELD OF THE INVENTION

The present invention relates generally to medical decision support systems and, more particularly, to computer-implemented platforms that integrate medical data governance, Internet-of-Things (IoT) medical device interoperability, and artificial-intelligence reasoning components, including large language models and modular AI agents, to generate governed, auditable, and scalable on-demand clinical insights.

### BACKGROUND OF THE INVENTION

Healthcare delivery increasingly depends on large volumes of heterogeneous digital data originating from electronic health record (EHR) systems, medical devices, diagnostic imaging systems, laboratory systems, and patient-generated data sources. Clinicians are expected to synthesize this information under time pressure, while complying with evolving regulatory frameworks, institutional policies, and payer requirements. At the same time, artificial-intelligence (AI) tools, including large language models, are being introduced into clinical workflows with limited governance, variable transparency, and inconsistent integration into existing information technology (IT) infrastructure.

Conventional clinical decision-support systems typically rely on static rules, preconfigured order sets, or narrowly scoped predictive models that are integrated with a single EHR vendor or device ecosystem. These systems often lack a robust medical data governance framework for normalizing and validating multi-source data, tracking provenance, or ensuring that only governed, patient-appropriate information is exposed to AI reasoning components. As a result, recommendations may be incomplete, inconsistent across care settings, or difficult to audit after the fact.

Existing AI-based healthcare tools further tend to operate as isolated applications, external chatbots, or vendor-specific modules that sit outside of the institution’s core governance architecture. In many cases, AI models are applied directly to raw or semi-structured data exported from clinical systems, without systematic enforcement of institutional policies, safety constraints, or cryptographic audit controls. This makes it difficult for providers to demonstrate that AI-assisted decisions were made using trustworthy data, in a manner consistent with local rules and regulatory expectations.

Applicant’s earlier work has described governance architectures and case-management systems that secure clinical data, enforce policy constraints, and maintain detailed audit trails for clinical episodes. However, there remains a need for a keystone platform that can sit above heterogeneous clinical systems and devices, expose a governed data abstraction to multiple AI reasoning components, orchestrate modular AI agents, and provide on-demand clinical insights at scale. There is also a need for such a platform to integrate networked medical devices and room infrastructure, support fast and slow reasoning pathways, and leverage cryptographic governance mechanisms to produce governed, auditable clinical recommendations.

### SUMMARY OF THE INVENTION

The present invention provides a computer-implemented AI keystone system that functions as a governed platform for advanced medical decision support and scalable on-demand clinical insights. In one aspect, the system includes a medical data governance (MDG) engine configured to ingest heterogeneous patient-related data from multiple clinical information systems, medical devices, and clinician inputs; validate, normalize, and timestamp that data under predefined governance rules; and generate structured, context-constrained data representations associated with identified patients and clinical episodes. These governed representations serve as the authoritative data substrate for downstream AI reasoning components.

In another aspect, the keystone system includes an open reasoning layer comprising one or more artificial-intelligence models, including at least one large language model (LLM), and a plurality of modular AI agents. A team-leader agent decomposes clinician queries into subtasks, routes those subtasks to specialized agents, and orchestrates their outputs. A validation or arbitration agent evaluates candidate clinical outputs against governance rules, institutional policies, and cryptographic audit constraints. A conversational-memory agent maintains longitudinal context across encounters, while additional agents manage metadata scoring, query optimization, and proactive task generation.

In certain embodiments, the keystone system further includes an IoT medical-device interoperability layer comprising wall-mounted medical hubs, active dongles, and edge-computing components that connect legacy and networked medical devices to the MDG engine. The interoperability layer may provide Power-over-Ethernet (PoE), local preprocessing, buffering, and wireless failover connectivity, and may convert native device signals into governed, standardized messages suitable for ingestion by the MDG engine.

In further aspects, the reasoning layer is organized into fast-thinking and slow-thinking components. A fast-thinking component operates on high-priority, governance-approved data subsets to produce low-latency insights, while a slow-thinking component performs deeper contextual analysis over broader governed datasets. An orchestrator merges these outputs into unified clinical recommendations while dynamically allocating computational resources, such as GPU capacity, between the components to improve efficiency without sacrificing clinical performance.

In additional embodiments, the keystone system incorporates a cryptographic governance subsystem configured to evaluate clinical outputs against patient-safety rules, regulatory mandates, institution-specific policies, and stored consent artefacts using machine-verifiable constraints; enforce dual-factor clinician authentication using secure hardware elements and possession factors; and maintain immutable, hash-chained audit records with trusted timestamps that gate advancement of workflow state. The system thereby produces governed, auditable clinical insights that can be traced back to specific governed data representations, AI agent operations, and governance checks, enabling defensible clinical use and regulatory review.

### BRIEF DESCRIPTION OF THE DRAWINGS

FIG. 1 is a schematic diagram of an example system architecture for cryptographic governance of clinical outputs, including a medical data governance (MDG) engine, an insight generation component, and a cryptographic governance subsystem.

FIG. 2 is a flowchart illustrating an example governance workflow for candidate clinical outputs, including authentication, policy-constraint evaluation, audit commitment, and controlled release to a clinician device.

FIG. 3 is a schematic diagram illustrating an example hash-chained audit trail and audit record structure used to provide immutable provenance and trusted timestamping for governed clinical outputs.

### DETAILED DESCRIPTION OF THE INVENTION

### System Overview

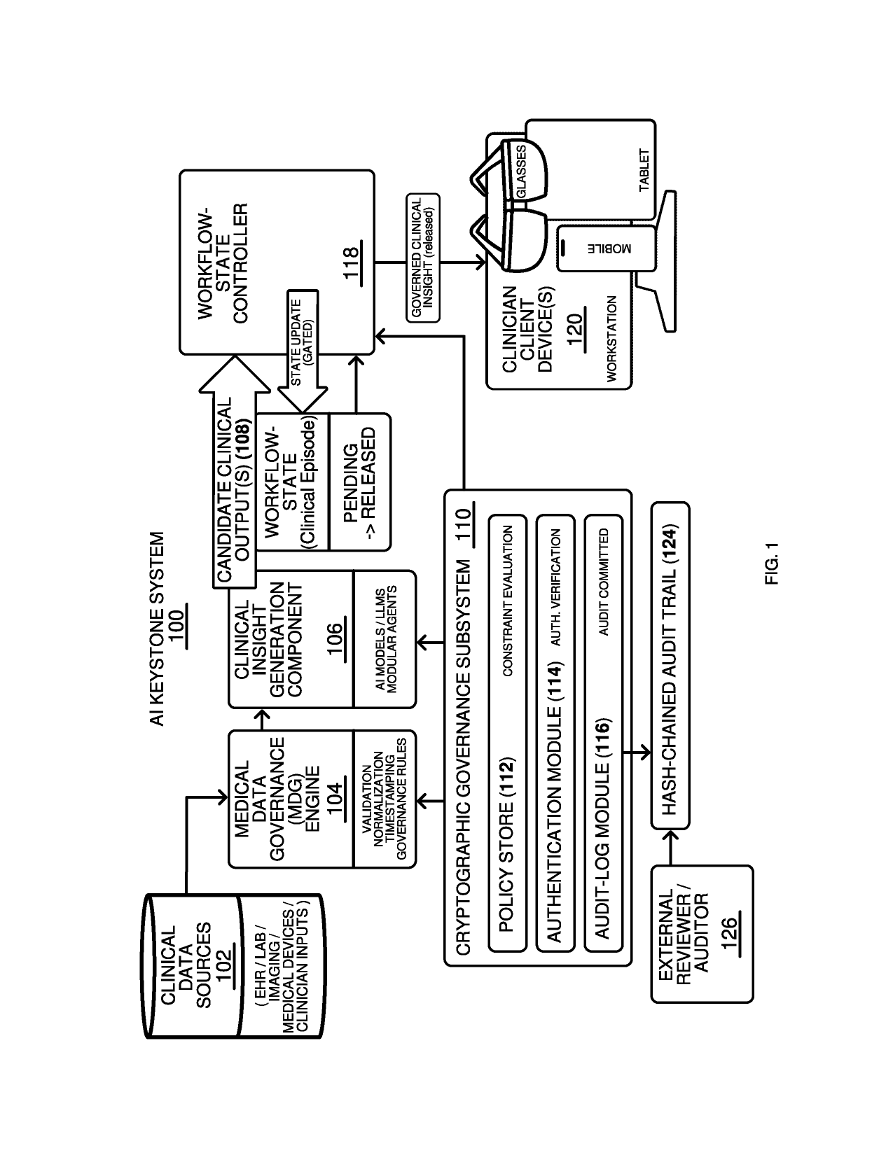

As shown in FIG. 1, an AI keystone system (100) receives raw patient-related data from clinical data sources (102), governs the data using a medical data governance (MDG) engine (104), generates candidate clinical outputs via an insight generation component (106), and governs release via a cryptographic governance subsystem (110) and a workflow-state controller (118) to clinician client device(s) (120). The embodiments described herein relate to an AI keystone system that provides governed clinical decision support and on-demand clinical insights by coordinating data governance, medical-device interoperability, artificial-intelligence reasoning, and cryptographic controls within a unified platform. While the following description is framed in the context of hospital, clinic, and virtual-care settings, the disclosed techniques may also be applied to ambulatory care, home monitoring, remote patient management, and other healthcare environments.

In general, the system includes a medical data governance (MDG) engine that ingests heterogeneous clinical data, an IoT and room-infrastructure layer that connects medical devices and sensors, a reasoning layer that incorporates one or more large language models (LLMs) and modular AI agents, and a cryptographic governance subsystem that enforces safety constraints, access control, and auditability. Together, these components form an AI keystone platform that exposes a governed, patient-centric data abstraction to multiple AI tools while maintaining institutional control over data quality, policy enforcement, and regulatory compliance.

The system can be deployed in a variety of configurations, including as on-premises software running in a hospital datacenter, as a hybrid deployment spanning on-premises equipment and cloud services, or as a fully cloud-hosted platform with secure connectivity to clinical information systems and medical devices. In some embodiments, core components of the MDG engine, cryptographic governance subsystem, and certain AI agents are deployed within a healthcare provider’s controlled infrastructure, while one or more LLMs are accessed through governed interfaces to external model providers.

For purposes of illustration, FIG. 1 depicts a representative system architecture in which the MDG engine receives data from electronic health record (EHR) systems, laboratory information systems, imaging archives, bedside monitors, infusion pumps, ventilators, and other clinical sources. The MDG engine validates, normalizes, and timestamps this data to create structured, context-constrained data representations that are associated with specific patients and clinical episodes. These governed representations are then exposed, via defined interfaces, to the reasoning layer and modular AI agents.

The reasoning layer orchestrates one or more AI models and agents to process clinician queries, generate candidate insights, and refine those insights under governance. In certain embodiments, the reasoning layer includes a team-leader agent that decomposes complex clinician questions into smaller subtasks; a conversational-memory agent that maintains longitudinal context across encounters; one or more domain-specific agents (for example, agents specialized in vital signs, medication management, diagnostics, or care coordination); and a validation or arbitration agent that checks candidate outputs against governance rules and cryptographic audit constraints.

The cryptographic governance subsystem may be implemented as one or more services that interface with the MDG engine and the reasoning layer. Among other functions, this subsystem enforces dual-factor clinician authentication, applies machine-verifiable safety and policy constraints to clinical outputs, maintains immutable audit trails for each clinical episode, and can gate progression of workflow states on successful completion of governance checks. In some embodiments, audit records are stored in hash-chained structures with trusted timestamps, enabling independent verification of the sequence and contents of clinical events.

Unless otherwise specified, the components and functions described herein may be implemented in software, hardware, or any combination thereof. Functional blocks are described for conceptual clarity and may be combined, split, or replicated across physical or virtual machines. The particular arrangement shown in the figures is one illustrative configuration, and those skilled in the art will recognize that other arrangements can be used without departing from the scope of the invention as defined by the claims.

### Medical Data Governance (MDG) Engine

With reference to FIG. 1, the MDG engine (104) ingests heterogeneous clinical data from data sources (102), applies governance rules to validate, normalize, and timestamp the data, and outputs governed context packages to the clinical insight generation component (106). In various embodiments, the AI keystone system includes a medical data governance (MDG) engine configured to ingest, normalize, validate, and manage heterogeneous clinical data originating from multiple sources. The MDG engine provides a governed, patient-centric data abstraction that insulates the reasoning layer and modular AI agents from the idiosyncrasies of underlying clinical systems and device protocols. The MDG engine may receive data from electronic health record (EHR) systems, laboratory information systems, radiology and imaging systems, pharmacy systems, bedside monitors, ventilators, infusion pumps, wearable sensors, and patient-reported data sources. Data may be received in a variety of formats, including HL7 v2 messages, FHIR resources, DICOM objects, proprietary device protocols, flat files, or streaming telemetry. The MDG engine maps these inputs into one or more internal canonical models, which may be implemented using relational schemas, document stores, graph representations, time-series databases, or combinations thereof.

In some embodiments, the MDG engine applies a multi-stage validation pipeline to incoming data. A first stage may perform syntactic validation (e.g., required fields present, basic type checks), while a second stage may perform semantic validation (e.g., unit consistency, range checks, code-system normalization, matching to known patient identities). A third stage may apply governance rules that consider consent artefacts, privacy preferences, institutional policies, and clinical context to determine whether particular data elements are available to specific AI agents or reasoning pathways. Data that fails validation or governance checks may be quarantined, flagged for manual review, or excluded from downstream processing.

The MDG engine may generate structured, context-constrained data representations that associate normalized data elements with a specific patient, encounter, location, and time interval. In certain embodiments, the MDG engine maintains a longitudinal patient record that includes time-aligned streams of vital signs, laboratory results, medication administrations, imaging findings, clinician notes, and device telemetry. Context-constrained representations may be defined for specific clinical episodes (e.g., an intensive care unit stay, a surgical procedure, a course of chemotherapy) and may include derived features, such as summary scores, risk estimates, and event annotations. In some embodiments, the MDG engine assigns metadata tags to governed data elements and aggregates, such as reliability scores, data-source provenance, freshness indicators, and governance status flags. These metadata tags can be used by modular AI agents to prioritize subsets of data when responding to a clinician query, to select between alternative data sources, or to adjust reasoning strategies based on data quality or completeness. For example, an AI agent may be configured to prefer laboratory values with higher provenance and quality scores, or to explicitly note when relevant data is stale or missing. The MDG engine may expose its governed data representations through one or more application programming interfaces (APIs) or service endpoints. In some embodiments, the APIs support both pull-based queries, in which the reasoning layer requests data for a specific patient and context, and push-based subscriptions, in which the MDG engine streams updates when new governed data elements become available. The MDG engine may further maintain an internal change-log that records additions, updates, and deletions of governed data, as well as governance decisions applied to particular data elements, thereby enabling traceability of the data used to support specific clinical insights.

### IoT Medical Device and Room Infrastructure

In FIG. 1, device-generated data may be included within the clinical data sources (102), and intermediary IoT components may preprocess and relay such data to the MDG engine (104) under governance constraints. In certain embodiments, the AI keystone system includes an Internet-of-Things (IoT) medical device and room-infrastructure layer that provides standardized connectivity between medical devices, sensors, and the MDG engine. This infrastructure enables plug-and-play integration of legacy and modern devices, reduces the configuration burden on clinical staff, and ensures that device data is captured, governed, and made available to the reasoning layer in a consistent manner.

As illustrated conceptually in FIG. 1, the IoT layer may include one or more wall-integrated medical hubs installed in patient rooms, procedure areas, or monitoring stations. Each hub may comprise a medical-grade enclosure with multiple physical ports (such as USB, serial, Ethernet, or specialized medical connectors) for attaching bedside monitors, ventilators, infusion pumps, and other devices. The hub may further include edge-computing circuitry, local storage or buffering memory, and network interfaces providing both wired and wireless connectivity. In some embodiments, the hub is powered via Power-over-Ethernet (PoE) and may provide power and data connectivity to connected devices. One or more active dongles or adapter cables may be used to connect devices that lack native network capabilities or that use proprietary signaling. Each dongle may include a microcontroller or system-on-chip configured to translate device-specific protocols into standardized messages understood by the hub, to apply basic validation, and to tag data with device identifiers and timestamps. In some embodiments, the dongles may also implement patient association logic, such as associating a physical device with a particular bed, room, or patient wristband through scanning, configuration, or automatic detection. The hubs may communicate with the MDG engine using secure, authenticated channels. In some embodiments, the hubs support both primary wired connections and secondary wireless networks, such that if the primary network link is disrupted, the hub can fail over to wireless connectivity while continuing to buffer and forward device data. Local buffering may allow the hub to capture and retain high-frequency telemetry or waveforms during transient network disruptions, and to transmit compressed summaries or segments to the MDG engine when connectivity is restored.

The IoT infrastructure may further support device discovery and configuration management. For example, when a new device is connected to a hub, the system may automatically identify the device based on a device profile, load appropriate protocol adapters or drivers, and register the device with the MDG engine. Device status, such as connectivity, battery level, and error conditions, may be monitored and reported to clinical and technical staff. In some embodiments, over-the-air updates for dongle firmware or hub software may be coordinated through the keystone platform to ensure that governance rules, security patches, and configuration policies remain current.

In various embodiments, the IoT layer supports the notion of a patient-centric clinical network. When a patient is admitted to a room or bed, the system may associate that patient with the local hub and attached devices, such that telemetry and measurements are automatically mapped to the correct patient context within the MDG engine. If the patient is moved to another location, the association may be updated, and the system may automatically reassign devices, reroute data streams, and update the governed patient record without requiring manual reconfiguration of each device. This patient-centric networking enables the keystone system to maintain continuous, governed visibility into device-derived data throughout the patient’s care journey.

### Reasoning Layer and LLM Interaction

With reference to FIG. 1, the clinical insight generation component (106) operates over governed context produced by the MDG engine (104) to generate candidate clinical outputs (108) for downstream governance and release. In various embodiments, the AI keystone system includes a reasoning layer that orchestrates one or more artificial-intelligence models and tools to generate clinical insights from governed data representations provided by the MDG engine. The reasoning layer may incorporate one or more large language models (LLMs), traditional machine-learning models, rules engines, search indices, and external clinical reference systems. The reasoning layer is configured to operate only on governed, context-constrained data, and to interact with that data through standardized interfaces that preserve auditability and policy enforcement.

In some embodiments, the reasoning layer exposes an application-facing interface that accepts clinician queries in natural language, structured form, or a combination thereof. Clinician queries may be submitted through an electronic health record (EHR) interface, a standalone application, a messaging client, a voice assistant, or an application programming interface (API). Queries may reference a specific patient, cohort, or clinical scenario, and may request tasks such as summarizing the patient’s status, identifying potential diagnoses or complications, checking guideline conformity, generating care-plan options, or prioritizing tasks for a case manager. Upon receiving a query, the reasoning layer may invoke a query-preprocessing component that performs operations such as language normalization, detection of key clinical entities, and identification of relevant temporal intervals. The preprocessed query may be transformed into one or more internal representations that are optimized for interaction with the MDG engine and the modular AI agents. In some embodiments, the reasoning layer generates a “data request specification” that describes what governed data is required, at what granularity, and under which governance constraints. The reasoning layer may then communicate with the MDG engine to retrieve governed, context-constrained data representations for the patient or cohort of interest. These representations may include structured data, such as vitals and laboratory values, as well as unstructured or semi-structured data, such as clinician notes, imaging reports, and device waveforms. In some embodiments, the reasoning layer performs iterative expansion and contraction of the data context: starting from a minimal set of high-value data and gradually expanding to broader context only when necessary to answer the query. Metadata tags and governance status indicators provided by the MDG engine may guide this process.

One or more LLMs may be invoked to interpret the clinician query, to propose candidate clinical insights, and to explain the rationale for those insights in natural language. The reasoning layer may enforce a strict separation between model-facing prompts and system control instructions, such that LLMs only see governed data and contextual instructions that have been vetted by the keystone platform. In some embodiments, the reasoning layer supplies the LLM with a structured context package containing selected data elements, metadata tags, and references to relevant guidelines or policies, together with a role-specific prompt that constrains the LLM’s behavior (e.g., focusing on summarization, comparison, or differential diagnosis rather than open-ended speculation).

The reasoning layer may also apply post-processing to LLM outputs, including schema validation, detection of hallucinations or unsupported statements, and alignment with governance rules. For example, the system may compare model-generated statements to the governed data context, flag statements that reference unsupported facts, and either correct or exclude such statements from the final output. In some embodiments, the reasoning layer may use secondary models or rules to assess the plausibility, safety, and completeness of candidate outputs before they are passed to modular AI agents for further processing or to the cryptographic governance subsystem for final validation. In certain implementations, the reasoning layer supports multiple LLMs and model providers, and may select among them based on task type, cost, latency, data sensitivity, and regulatory requirements. For example, a locally hosted model may be used for highly sensitive patient data, whereas an external hosted model may be used for de-identified or synthetic data or for tasks with less stringent latency constraints. The keystone system may maintain routing policies and performance metrics that allow the reasoning layer to choose appropriate models and to adapt over time as model capabilities and institutional requirements evolve.

### Modular AI Agents and Collaboration

In some embodiments, candidate clinical outputs (108) generated by the insight generation component (106) are further refined or evaluated by cooperating agents prior to controlled release as governed clinical insights to clinician client device(s) (120) as depicted in FIG. 1. In various embodiments, the reasoning layer is augmented by a set of modular AI agents that collaborate to process clinician queries, manage context, and produce governed clinical insights. These agents may be implemented as software services, stateful workflows, or specialized prompts and control structures around one or more LLMs. By decomposing responsibilities across multiple agents, the keystone system can support complex clinical reasoning tasks while maintaining clear separation of concerns and traceability of each agent’s contribution.

A team-leader agent may serve as an overall coordinator for a given clinician query or episode of interaction. The team-leader agent can analyze the query, identify the subtasks required to answer it, and construct a task graph specifying dependencies between subtasks. Subtasks may include retrieving specific governed data from the MDG engine, checking guideline conformity, evaluating risk scores, summarizing status across body systems, or identifying missing information that should be collected. The team-leader agent may assign these subtasks to other agents, monitor their progress, and integrate their outputs into a coherent intermediate or final response. A conversational-memory agent may maintain longitudinal context for ongoing interactions with a clinician about a particular patient or set of patients. The conversational-memory agent can store and retrieve dialogue history, previous system recommendations, clinician decisions, and unresolved tasks. In some embodiments, the memory agent maintains both short-term context (e.g., the last several turns in a conversation) and structured long-term context (e.g., a summary of a patient’s course over time). The agent may use this context to help other agents interpret new queries, avoid redundant suggestions, and surface relevant prior insights or decisions.

A validation or arbitration agent may be responsible for assessing candidate outputs generated by other agents or by LLMs against governance rules, institutional policies, and cryptographic constraints. For example, the validation agent may check that medication suggestions are compatible with the patient’s current medications and allergies, that diagnostic recommendations do not contradict critical lab results, and that all outputs are traceable to governed data elements. The validation agent may either approve candidate outputs, request additional information, or generate alternative recommendations when conflicts are detected. In some embodiments, the validation agent can reconcile divergent outputs from multiple specialized agents or models and produce a consensus recommendation.

One or more metadata agents may compute and update scores related to data quality, relevance, and governance status. These agents can evaluate the completeness of the governed data context for a particular query, identify gaps or inconsistencies, and assign confidence scores to various hypotheses or recommendations. Metadata agents may also monitor system performance indicators, such as latency, compute utilization, and error rates, and provide feedback to other agents or orchestration components to adjust their strategies (for example, choosing simpler reasoning pathways when latency constraints are strict).

A query-optimization agent may transform high-level clinician questions into structured requests and function calls that can be executed by the MDG engine and other system components. For instance, the query-optimization agent may map natural-language questions to parameterized templates, select appropriate patient cohorts for population-level analyses, or construct time-windowed queries for longitudinal data. The agent may also normalize terminology to standard code systems and ontologies, thereby enabling consistent matching and retrieval across heterogeneous data sources. In some embodiments, additional specialized agents may be provided for particular domains or workflows, such as medication management, sepsis detection, perioperative care, oncology pathways, or discharge planning. These specialized agents may encapsulate domain-specific rules, models, or guidelines and may be invoked selectively by the team-leader agent when relevant. Each agent may expose an interface for receiving task specifications and returning structured outputs, and the keystone platform may maintain logs that record which agents were invoked, the inputs they received, and the outputs they generated. The collaboration between agents may be organized using standard task-graph or message-passing patterns. In all cases, the keystone system may record agent interactions and intermediate states as part of a governance trace, such that for any final clinical insight presented to a user, it is possible to reconstruct which agents contributed, what data they used, and what intermediate reasoning steps occurred. This agent-level traceability supports audit, debugging, and continuous improvement of the system, and can be used to satisfy regulatory and institutional requirements for explainability and accountability.

### Fast-Thinking and Slow-Thinking Components; Resource Optimization

In various embodiments, the insight generation component (106) may execute fast-response and deliberative reasoning modes, while release of any candidate clinical outputs (108) remains gated by the workflow-state controller (118) and associated governance mechanisms shown in FIG. 1. In various embodiments, the AI keystone system organizes certain reasoning workflows into fast-thinking and slow-thinking components that operate over the governed data representations provided by the MDG engine. This separation enables the system to deliver low-latency insights when time is critical, while still supporting deeper, more computationally intensive analyses when circumstances permit.

A fast-thinking component may be configured to operate on a focused subset of high-priority, governance-approved data elements selected for their relevance to common time-sensitive tasks such as triage, early warning, and safety checks. For example, the fast-thinking component may prioritize recent vital signs, key laboratory results, active medications, allergies, and critical device alarms. Using simplified models, rule sets, or LLM prompts constrained to summarization and pattern recognition, the fast-thinking component can quickly generate outputs such as risk flags, concise status summaries, and alerts for potential deterioration or missed actions.

A slow-thinking component may be configured to operate on broader governed datasets and to support more complex reasoning tasks, such as differential diagnosis, longitudinal trend analysis, care-plan optimization, or population-level cohort analysis. The slow-thinking component may leverage richer context, including extended clinical histories, imaging and pathology reports, procedural records, and longitudinal device telemetry. It may also employ more computationally expensive models or multi-step agent collaborations to explore alternative hypotheses, quantify uncertainty, or simulate outcomes under different interventions.

In some embodiments, an orchestrator module manages the interaction between fast-thinking and slow-thinking components. The orchestrator may route a given clinician query first to the fast-thinking component to obtain an initial low-latency response, and then, when appropriate, supplement that response with additional insights from the slow-thinking component as they become available. The orchestrator may also determine when it is sufficient to use only fast-thinking pathways, such as for simple clarifications or quick checks, and when to escalate to slow-thinking pathways, such as for complex or high-risk cases.

The keystone system may further implement resource-optimization mechanisms to manage compute, memory, storage, and network bandwidth. In resource-constrained or cost-sensitive environments, the keystone system may employ tiered reasoning strategies. For instance, a small, locally hosted model may be used as a default for most queries, with larger or external models invoked only when the expected benefit justifies the additional cost or latency. The system may track historical performance metrics and clinical feedback to refine these strategies over time, such that commonly recurring queries are routed along efficient, well-characterized pathways, while novel or high-impact queries receive more intensive analysis. In all cases, the separation between fast-thinking and slow-thinking components, combined with explicit orchestration and governance, allows the keystone system to balance responsiveness, depth of reasoning, and resource utilization.

### Cryptographic Governance Subsystem and Audit Trails

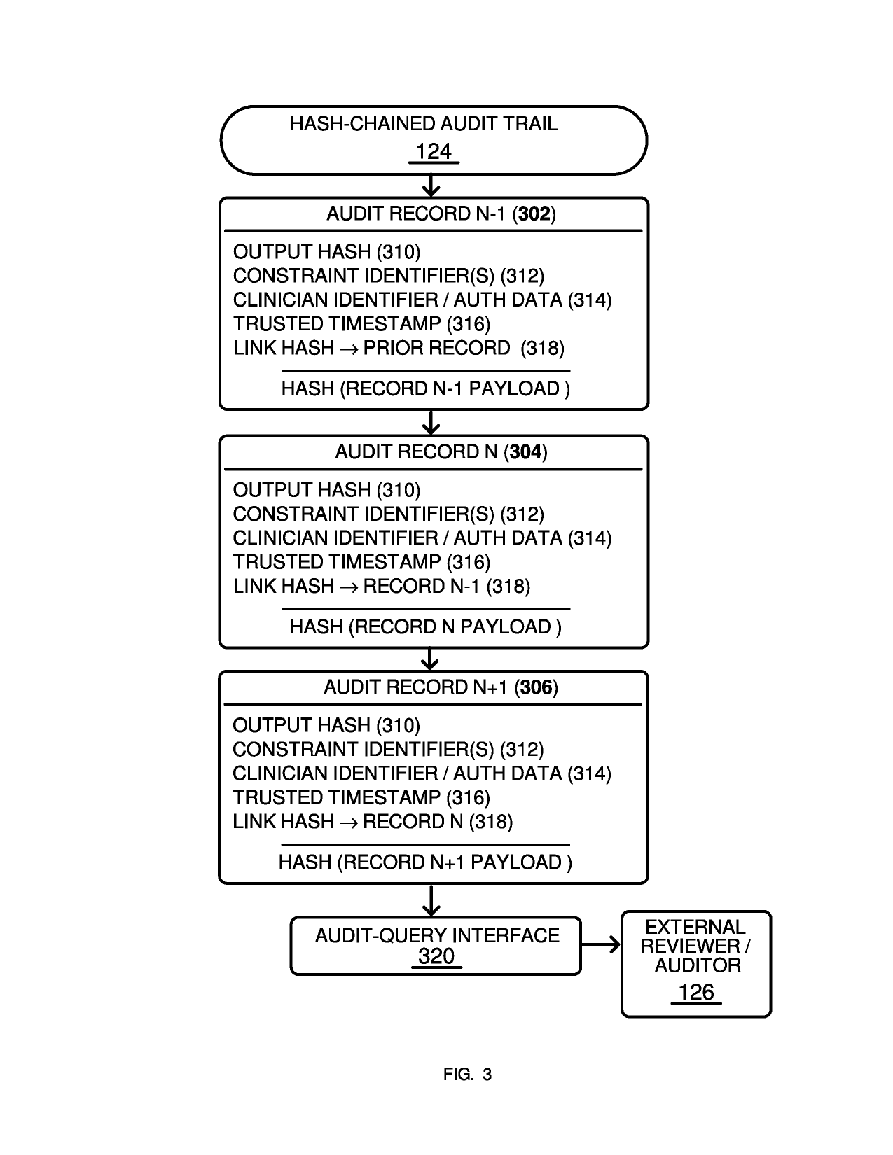

As shown in FIG. 1, the cryptographic governance subsystem (110) may include a policy store (112), an authentication module (114), and an audit-log module (116) that collectively support workflow-state gating by the workflow-state controller (118) and controlled delivery to clinician client device(s) (120); immutable audit records may be stored in a hash-chained audit trail (124) as further illustrated in FIG. 3. In various embodiments, the AI keystone system includes a cryptographic governance subsystem that enforces safety constraints, access control, and auditability across data ingestion, reasoning, and insight delivery workflows. The cryptographic governance subsystem provides guarantees that clinical outputs are generated and delivered in accordance with patient-safety rules, regulatory mandates, institution-specific policies, and recorded patient consents, and that the sequence of relevant events can be independently verified.

The cryptographic governance subsystem may interface with the MDG engine, the reasoning layer, and external identity and access-management systems. Among other functions, it may enforce dual-factor authentication for clinician users, such that access to high-impact actions or sensitive insights requires both a biometric factor (for example, fingerprint or facial recognition) executed in a secure hardware element, and a possession factor (for example, a security token or mobile device) associated with the clinician’s identity. The subsystem may further apply time-boxed access scopes, limiting the duration and extent of access to particular patients, units, or functions based on role, context, and policy. The subsystem may encode patient-safety rules, institutional policies, and regulatory constraints as machine-verifiable governance rules that can be applied to candidate clinical outputs. For example, governance rules may specify maximum allowable dose ranges, contraindicated medication combinations, escalation thresholds for abnormal vital signs, documentation requirements for certain interventions, or restrictions on jurisdictional or institutional data-handling requirements. When the reasoning layer or modular AI agents generate candidate insights or recommendations, the cryptographic governance subsystem may evaluate those outputs against the rule set and either approve them, modify them, or block them. In some embodiments, blocked outputs may be accompanied by explanations or alternative, compliant recommendations.

In addition, the cryptographic governance subsystem may maintain immutable audit trails that record key events for each clinical episode, including data access, authentication events, governance decisions, and the generation and delivery of clinical insights. Audit records may be stored in hash-chained structures, such as hash-linked logs, that allow the integrity and ordering of entries to be verified. Each record may include a trusted timestamp, a representation of the governed data or insight to which it relates, references to the AI agents or models involved, and identifiers for the users or systems that initiated or received the action.

In some embodiments, advancement of certain workflow states may be gated on successful governance checks. For example, releasing a high-risk medication recommendation, closing a critical task, or transmitting data to an external system may require that the corresponding audit entry be committed to the hash-chained log and that all applicable governance rules be satisfied. If governance checks fail, the system may prevent the workflow from advancing and may provide guidance on remediation steps, such as obtaining additional data, adjusting parameters, or seeking human override by an appropriately authorized clinician.

The cryptographic governance subsystem may expose interfaces for internal and external audit, allowing authorized parties to query, verify, and analyze governance traces for particular patients, episodes, or time intervals. These traces may be used for quality improvement, incident investigation, regulatory reporting, and legal or compliance reviews. By tying clinical insights to verifiable governance events and immutable audit records, the keystone system enables healthcare organizations to demonstrate how AI-assisted decisions were made, what data and policies were involved, and which human actors were responsible for oversight and execution.

### Governance Workflow for Candidate Clinical Outputs

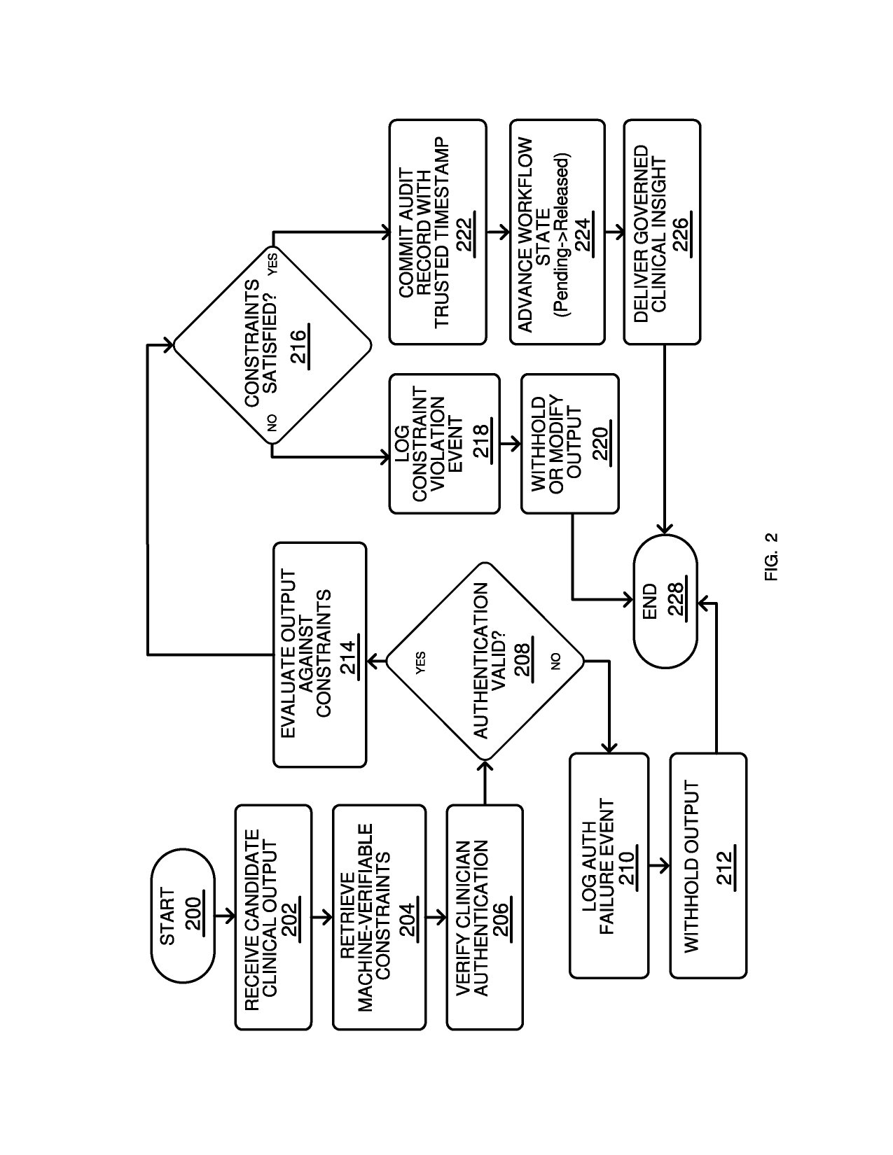

As shown in FIG. 2, a governance workflow begins at start node (200) and receives a candidate clinical output at step (202) from the clinical insight generation component (106). At step (204), applicable machine-verifiable constraints are retrieved from the policy store (112). At step (206), clinician dual-factor authentication is verified via the authentication module (114) and a time-boxed access scope may be derived. At decision (208), if authentication or scope is invalid, the event is logged at step (210) to the audit-log module (116) and the workflow ends with the output withheld at node (212). If authentication is valid, the workflow evaluates the candidate output against constraints at step (214) and determines at decision (216) whether constraints are satisfied. If constraints are not satisfied, a violation record is logged at step (218) to the audit-log module (116) and the workflow ends with the output withheld or modified at node (220). If constraints are satisfied, an audit record is committed at step (222) to the hash-chained audit trail (124) with a trusted timestamp, the workflow state is advanced at step (224) (e.g., pending to released), and the governed clinical insight is delivered at step (226) to clinician client device(s) (120), ending at node (228).

In this example, steps (204)-(222) correspond to constraint evaluation, authentication verification, and audit-record commitment that gate advancement of workflow state by the workflow-state controller (118) prior to delivery of the governed clinical insight.

### Hash-Chained Audit Trail Record Structure

As shown in FIG. 3, the hash-chained audit trail (124) comprises a sequence of audit records including audit record N-1 (302), audit record N (304), and audit record N+1 (306), where each audit record is cryptographically linked to at least a portion of a preceding audit record to provide integrity and tamper evidence. In one example, audit record N (304) includes an output hash field (310) representing a hash of at least a portion of a candidate clinical output, including when released, a constraint identifier field (312) identifying one or more machine-verifiable constraints evaluated by the policy store (112), a clinician identifier and authentication information field (314) associated with the authenticated clinician identity, a trusted timestamp field (316), and a link hash field (318) that cryptographically links audit record N (304) to at least a portion of audit record N-1 (302).

In the illustrated embodiment, the audit-log module (116) appends audit records to the hash-chained audit trail (124) upon completion of governance checks, and the workflow-state controller (118) gates advancement of a workflow state associated with a clinical episode based on commitment of an audit record to the hash-chained audit trail (124). An audit-query interface (320) may retrieve and validate trace evidence for a clinical episode by verifying hash linkage and trusted timestamps across one or more audit records, and an external reviewer or auditor (126) may interact with the audit-query interface (320) to request and validate such trace evidence.

### Example Workflow: Intensive Care Unit Deterioration Detection

In one illustrative example, the AI keystone system is deployed in an intensive care unit (ICU) to assist in early detection of patient deterioration. Each ICU bed is equipped with a wall-integrated IoT medical hub that connects bedside monitors, ventilators, infusion pumps, and other devices. When a patient is admitted or transferred into the bed, the patient’s identity is associated with the hub and connected devices, such that forthcoming telemetry and measurements are mapped to the correct patient context within the MDG engine. As devices stream vital signs, ventilator parameters, infusion rates, and alarms, the IoT hubs buffer, normalize, and transmit data to the MDG engine using secure, authenticated channels. The MDG engine validates the incoming data, resolves patient identity, normalizes units and codes, and updates the longitudinal patient record with time-aligned streams of governed data. The MDG engine also assigns metadata such as data-source provenance, freshness, and governance status, making this information available to the reasoning layer. A clinician may submit a query such as “Is this patient at risk of deterioration over the next 6–12 hours?” through an EHR-integrated interface or bedside workstation. The reasoning layer receives the query, performs preprocessing to extract key entities and time horizons, and generates a data request specification that identifies recent vital signs, laboratory trends, active medications, ventilator settings, and relevant comorbidities as required inputs. The MDG engine returns governed, context-constrained data for the patient, including any applicable risk scores or event annotations. A fast-thinking component may first operate on a narrow subset of high-priority data (e.g., last several hours of vitals, most recent labs, current ventilator settings) to produce a low-latency status summary and immediate risk flag. Modular AI agents may identify acute abnormalities, missing data that should be obtained, or guideline-based thresholds that have been crossed. A team-leader agent orchestrates these subtasks and forwards the provisional findings to a validation or arbitration agent.

In parallel or subsequently, a slow-thinking component may operate on a broader governed dataset spanning days or weeks of clinical history. This component may examine longer-term trends, prior episodes of instability, treatment responses, and comorbidity patterns. It may simulate multiple scenarios (such as escalation of respiratory support or initiation of specific therapies) and estimate the likelihood of deterioration under different care plans. Outputs from the slow-thinking component are integrated with the fast-thinking summary under the control of the team-leader agent.

Before any recommendation is delivered to the clinician, the cryptographic governance subsystem evaluates candidate outputs against patient-safety rules, institutional policies, and applicable consent artefacts. For example, the subsystem may check that recommended medication changes do not violate dose limits or known contraindications and that suggested interventions are supported by the available governed data. Approved insights are logged into an immutable audit trail with trusted timestamps and references to the governed data and agents involved. The final, governed clinical insight - such as a risk stratification label, a prioritized list of concerns, and suggested evaluation or monitoring steps -is then delivered to the clinician with appropriate explanations and provenance indicators.

### Example Workflow: Virtual Ward and Remote Monitoring

In another example, the AI keystone system is used to support a “virtual ward” in which patients discharged from the hospital or clinic are monitored remotely using connected devices and periodic check-ins. Patients may be equipped with home blood-pressure cuffs, pulse oximeters, weight scales, glucometers, or wearable sensors that transmit measurements through patient gateways or mobile applications. These gateways function similarly to the in-hospital IoT hubs, translating device signals into standardized, authenticated messages and forwarding them to the MDG engine.

The MDG engine ingests remote monitoring data alongside traditional clinical data, such as recent discharge summaries, medication lists, laboratory results, and follow-up appointments. For each enrolled patient, the MDG engine maintains a longitudinal record that integrates remote measurements with in-person encounters, and assigns metadata reflecting data recency, adherence patterns, and signal quality. Governance rules may specify which data elements can be used for particular types of remote insights and which must be reviewed by human clinicians before actions are taken.

Clinicians or care coordinators may issue queries such as “Which virtual ward patients are at highest risk of readmission in the next week?” or “Summarize changes in status since discharge for this patient.” A query-optimization agent may transform these high-level questions into structured cohort-selection and time-windowed queries. The MDG engine returns governed data for the relevant patients, including remote device trends, symptom reports, adherence indicators, and prior risk scores.

A fast-thinking component may rapidly scan incoming data streams to detect threshold breaches or sudden changes, such as sharp increases in weight suggestive of fluid overload, sustained desaturation events, or missed medication doses. Alerts generated by the fast-thinking component may be routed to care coordinators for timely follow-up. At the same time, a slow-thinking component may periodically perform deeper analyses across the virtual ward population, identifying patients whose risk trajectories are worsening, whose engagement with monitoring is declining, or whose care plans may warrant adjustment.

Modular AI agents may collaborate to generate patient-level summaries and prioritized worklists. For example, a conversational-memory agent may maintain context about each patient’s discharge plan, prior recommendations, and recent contacts, while a specialized care-coordination agent suggests specific outreach actions, such as scheduling a telehealth visit, adjusting monitoring frequency, or reinforcing self-care education. A validation or arbitration agent may ensure that suggested actions conform to program policies, local regulations, and any applicable reimbursement criteria before they are surfaced to staff.

The cryptographic governance subsystem records key events in an immutable audit trail, including receipt of device data, detection of risk signals, generation of recommendations, and clinician responses. For instance, if the system recommends that a nurse call a patient to check for worsening symptoms, the recommendation and subsequent documentation of the call can be linked within the governance trace. These records support program evaluation, regulatory reporting, and continuous improvement of the virtual ward model, while providing transparency into how AI-assisted insights contributed to patient management.

What is claimed is:

1. A computer-implemented system for cryptographic governance of clinical outputs generated by automated reasoning processes, comprising:
(a) a clinical insight generation component configured to receive patient-related data that has been validated and normalized under predefined governance rules and to generate one or more candidate clinical outputs associated with an identified patient and clinical episode;
(b) a cryptographic governance subsystem operatively coupled to the clinical insight generation component, the cryptographic governance subsystem comprising:
(i) a policy store configured to maintain machine-verifiable constraints representing patient-safety rules, regulatory mandates, institution-specific policies, and stored patient-consent artefacts;
(ii) an authentication module configured to enforce dual-factor authentication for clinicians, including a biometric factor executed in a secure hardware element and a possession factor associated with a clinician identity; and
(iii) an audit-log module configured to maintain an immutable audit trail of clinical episodes using hash-chained records with trusted timestamps; and
(c) a workflow-state controller configured to:
(i) evaluate each candidate clinical output against the machine-verifiable constraints maintained in the policy store;
(ii) upon determining that the candidate clinical output satisfies the machine-verifiable constraints and that a clinician has been authenticated by the authentication module, commit an audit record describing the candidate clinical output to the hash-chained audit trail with a trusted timestamp; and
(iii) gate advancement of a workflow state associated with the clinical episode from a pending state to a released state based at least on successful commitment of the audit record to the hash-chained audit trail.

2. The system of claim 1, wherein each hash-chained record in the audit trail comprises:
(a) a cryptographic hash of at least a portion of a payload describing the candidate clinical output, including when released;
(b) a reference to one or more identifiers of machine-verifiable constraints evaluated by the workflow-state controller;
(c) an identifier of a clinician authenticated by the authentication module; and
(d) a link value comprising a cryptographic hash of at least a portion of a preceding audit record in the audit trail.

3. The system of claim 1, wherein the authentication module is further configured to assign, upon successful dual-factor authentication, a time-boxed access scope specifying at least one of a patient, a clinical location, or a clinical role, and wherein the workflow-state controller is configured to prevent advancement of the workflow state to the released state if the time-boxed access scope has expired or does not cover the identified patient.

4. The system of claim 1, further comprising an audit-query interface configured to provide, to an authorized external reviewer, verifiable evidence of governance for a released clinical output by exposing at least a portion of the hash-chained audit trail corresponding to the clinical episode, including the trusted timestamp and identifiers of machine-verifiable constraints evaluated prior to advancement of the workflow state.
